(19) United States
(12) Patent Application Publication
(10) Pub. No.: US 2006/0095067 A1
(43) Pub. Date: May 4, 2006

(54) Lubricious filter

(76) Inventors: Horng-Ban Lin, Maple Grove, MN (US); James G. Hansen, Coon Rapids, MN (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 10/979,000

(22) Filed: Nov. 1, 2004

(51) Int. Cl. A61M 29/00

(52) U.S. Cl. 606/200

(57) ABSTRACT

Intravascular filters can include a filter membrane having a hydrophilic polymer coating on the filter membrane. The hydrophilic polymer coating can be on an inner surface, an outer surface or both an inner surface and an outer surface of the filter membrane. Intravascular filters including a hydrophilic polymer coating may be considered as providing increased hemocompatibility, decreased risk of filter-induced thrombosis and reduced sheathing forces.

### TECHNICAL FIELD

The invention relates generally to intravascular devices and more particularly to emboli-capturing devices. In particular, the invention relates to lubricious emboli-capturing devices such as filters.

### BACKGROUND

Heart and vascular disease are major problems in the United Sates and throughout the world. Conditions such as atherosclerosis result in blood vessels becoming blocked or narrowed. This blockage can result in lack of oxygenation of the heart, which has significant consequences since the heart muscle must be well oxygenated in order to maintain its blood pumping action.

Occluded, stenotic or narrowed blood vessels may be treated with a number of relatively non-invasive medical procedures including percutaneous transluminal angioplasty (PTA), percutaneous transluminal coronary angioplasty (PTCA), and atherectomy. Angioplasty techniques such as PTA and PTCA typically involve the use of a balloon catheter. The balloon catheter is advanced over a guidewire such that the balloon is positioned adjacent a stenotic lesion. The balloon is then inflated, and the restriction in the vessel is opened. During an atherectomy procedure, the stenotic lesion may be mechanically or otherwise cut away from the blood vessel wall using an atherectomy catheter.

During procedures such as angioplasty and atherectomy procedures, embolic debris can be separated from the wall of the blood vessel. If this debris enters the circulatory system, it can block other vascular regions including the neural and pulmonary vasculature. During angioplasty procedures, stenotic debris may also break loose due to manipulation of the blood vessel.

Because of this debris, a number of devices such as intravascular filters have been developed. A need remains for improved intravascular filters and filter membranes. A need remains for improved method of manufacture of intravascular filters and filter membranes.

### SUMMARY

The present invention is directed to improved intravascular filters and methods of manufacture thereof. In particular, the present invention is directed to intravascular filters that include a filter membrane and a hydrophilic polymer coating disposed on the filter membrane. The hydrophilic polymer coating can be on an inner surface, an outer surface or both an inner surface and an outer surface of the filter membrane. The present invention is directed to intravascular filters that in some embodiments may be considered as possessing increased hemocompatibility and posing a reduced risk of filter-induced thrombosis. In some cases, the present invention is directed to intravascular filters that may be considered as providing reduced sheathing forces.

Accordingly, an example embodiment of the invention can be found in a method of forming an intravascular filter that includes a polymeric base layer and a hydrophilic layer disposed on the base layer. A filter forming mandrel is provided, and the filter forming mandrel is sprayed with a hydrophilic polymer to form the hydrophilic layer. Subsequently, the hydrophilic layer is sprayed with a base polymer to form the base layer.

Another example embodiment of the invention can be found in a method of forming an intravascular filter that includes a polymeric base layer and a hydrophilic layer disposed on the base layer. A filter forming mandrel is provided, and the filter forming mandrel is sprayed with a base polymer to form the base layer. Subsequently, the base layer is sprayed with a hydrophilic polymer to form the hydrophilic layer.

Yet another example embodiment of the invention can be found in an intravascular filter that includes a filter membrane having an inner surface and an outer surface. A hydrophilic polyurethane coating can be disposed on the filter membrane on at least one of the inner surface of the filter membrane and the outer surface of the filter membrane.

The above summary of the present invention is not intended to describe each disclosed embodiment or every implementation of the present invention. The Figures, Detailed Description and Examples which follow more particularly exemplify these embodiments.

### BRIEF DESCRIPTION OF THE FIGURES

The invention may be more completely understood in consideration of the following detailed description of various embodiments of the invention in connection with the accompanying drawings, in which:

FIG. 1 is a schematic perspective view of an intravascular filter in accordance with an embodiment of the invention;

FIG. 2 is a magnified view of a portion of the filter membrane included in the intravascular filter of FIG. 1;

FIG. 3 is a schematic illustration of a filter forming mandrel and spray apparatus in accordance with an embodiment of the invention;

FIG. 4 is a schematic illustration of the apparatus of FIG. 3, including a first layer formed on the filter forming mandrel;

FIG. 5 is a schematic illustration of the apparatus of FIG. 3, including a second layer formed on the filter forming mandrel;

FIG. 6 is a schematic perspective view of a filter membrane made by the method illustrated in FIGS. 3-6; and

FIG. 7 is a cross-section taken along line 7-7 of FIG. 6.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value, i.e., having the same function or result. In many instances, the term “about” may include numbers that are rounded to the nearest significant figure.

The recitation of numerical ranges by endpoints includes all numbers within that range. For example, a range of 1 to 5 includes 1, 1.5, 2, 2.75, 3, 3.80, 4 and 5.

As used in this specification and in the appended claims, the singular forms “a”, “an”, and “the” include plural referents unless the content clearly dictates otherwise. As used in this specification and in the appended claims, the term “or” is generally employed in its sense including “and/or” unless the content clearly dictates otherwise.

The following description should be read with reference to the drawings, in which like elements in different drawings are numbered in like fashion. The drawings, which are not necessarily to scale, depict selected embodiments and are not intended to limit the scope of the invention. Although examples of construction, dimensions, and materials are illustrated for the various elements, those skilled in the art will recognize that many of the examples provided have suitable alternatives that may be utilized.

A hydrophilic polymer is a polymer that attracts or binds water molecules when the polymer is placed in contact with an aqueous system. Examples of aqueous systems that can provide water molecules that can bind to a hydrophilic polymer include blood and other bodily fluids. When a hydrophilic polymer comes into contact with such a system, water molecules can bind to the polymer via mechanisms such as hydrogen bonding between the water molecules and substituents or functional groups present within or on the polymer. In some instances, a hydrophilic polymer can bind at least 2 times its own weight in water and in particular instances some hydrophilic polymers can bind up to about 20 times their own weight in water.

One class of polymers that can be considered as hydrophilic includes certain nonionic polymers such as hydrophilic polyurethanes. Examples of suitable materials include nonionic polyether polyurethanes available commercially under the HYDROSLIP® name. Another suitable material includes nonionic aliphatic polyether polyurethanes available commercially under the TECOGEL® name. Examples of other suitable nonionic polymers include polymers such as poly (hydroxy methacrylate), poly (vinyl alcohol), poly (ethylene oxide), poly (n-vinyl-2-pyrolidone), poly (acrylamide) and other similar materials.

Another class of polymers that can be considered as hydrophilic includes ionomer polymers. An ionomer polymer is a polymer that has includes charged functional groups. The charged functional groups can be positively charged, in which case the polymer can be referred to be a cationomer, or the functional groups can be negatively charged, in which case the polymer can be referred to as an anionomer.

An ionomeric polymer can be formed using a variety of negatively charged functional groups. The negatively charged functional group can be added to a previously formed polymer, or the negatively charged functional groups can be part of one or more of the monomers used to form the ionomeric polymer.

Examples of suitable negatively charged functional groups include sulfonates and carboxylates. The ionomeric polymer can, in particular, include sulfonate functional groups. These groups are negatively charged and can readily hydrogen bond sufficient amounts of water when brought into contact with a source of water such as an aqueous system. Additional examples of ionomeric polymers include poly (acrylic acid), poly (methacrylic acid), hydroluronic acid, collagen, and other similar materials.

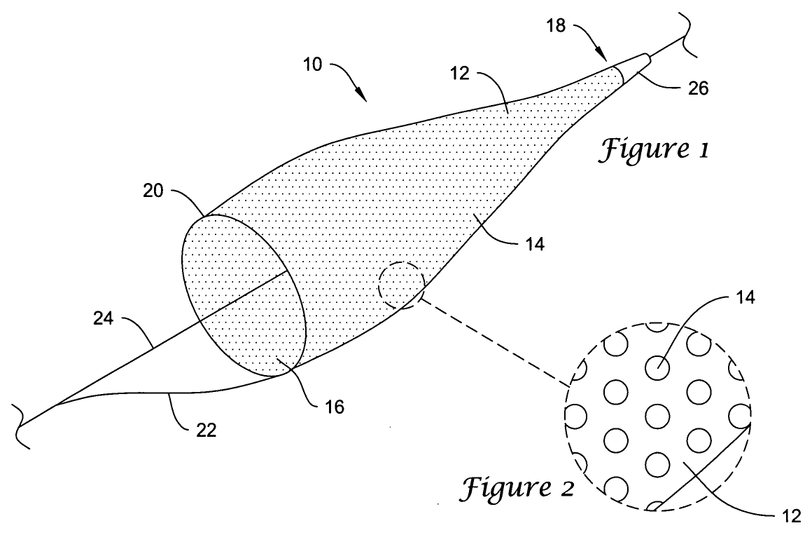

Turning now to the Figures, FIG. 1 is a perspective view of an example intravascular filter 10, which includes a filter membrane 12. The filter membrane 12 can be formed from any suitable material or combination of materials as will be discussed in greater detail hereinafter. The filter membrane 12 can be porous, having pores 14 that are configured to permit blood flow while retaining embolic material of a desired size. The filter membrane 12 can have a mouth 16 and a closed end 18 and is capable of moving between an open state and a closed state. The mouth 16 can be sized to occlude the lumen of the body vessel in which the filter may be installed, thereby directing all fluid and any emboli into the filter with emboli retained therein.

A support hoop 20 can be attached to the filter membrane 12 at or proximate to the mouth 16. The support hoop 20 can be attached to the filter membrane 12 through melt bonding or other suitable means. In some embodiments, as discussed in greater detail hereinafter, the support loop 20 can be integrally molded within the filter membrane 12. The support hoop 20 has an expanded state and a compressed state. The expanded state of the support hoop 20 is configured to urge the mouth 16 to its full size, while the compressed state permits insertion into a small lumen.

The support hoop 20 can be made from a flexible metal such as spring steel, from a super-elastic elastic material such as a suitable nickel-titanium alloy, or from other suitable material. The support hoop 20 can be a closed hoop made from a wire of uniform diameter, it can be a closed hoop made from a wire having a portion with a smaller diameter, it can be an open hoop having a gap, or it can have another suitable configuration.

A strut 22 can be fixedly or slideably attached to and extend from the support hoop 20. An elongate member 24 can be attached to and extend from the strut 22. The elongate member 24 can be attached to the strut 22 at an angle or the strut 22 can have a small bend, either at a point or over a region. The strut 22 can be attached to the support hoop 20 at a slight angle such that when the elongate member 24, the strut 22, and the support hoop 20 are in an unconstrained position, the elongate member 24 can generally extend perpendicular to the support hoop 20.

In the unconstrained position, the elongate member 24 can also lie along an axis which passes through the center of the region created by the support hoop 20. This may help position the support hoop 20 in contact with the wall of a vascular lumen or it may help in enhancing predictability or reliability during deployment. In some embodiments, the elongate member 24 can terminate at the strut 22. In other embodiments, the elongate member 24 can extend through the filter membrane 12, as shown. Whether or not the elongate member 24 extends through the filter membrane 12, it may be fixedly or slideably/rotatably attached to the filter membrane 12.

The filter membrane 12 can include a waist 26 at a closed end 28. In some embodiments, the waist 26 can be integrally formed with the filter membrane 12. In other embodiments, the filter membrane 12 can be further processed to form the waist 26. In some embodiments, integrally forming the waist 26 with the filter membrane 12 can reduce the outer diameter of the filter device when in a compressed state, increase the reliability and uniformity of the bond between the filter membrane and the elongate member, and reduce the number of steps or components needed to form the filter device.

The waist 26 is a region largely incapable of moving between two states and having a lumen of substantially constant diameter therethrough. The elongate member 24 can extend through and be bonded to the waist 26. This bonding can be heat bonding such as laser bonding, or may be an adhesive or other suitable means.

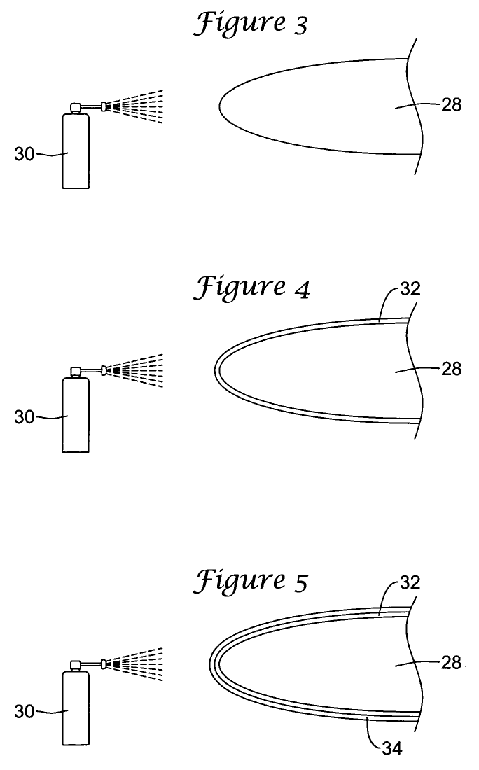

FIGS. 3 through 5 illustrate exemplary methods of forming the filter membrane 12 in accordance with the invention. FIG. 3 shows a filter forming mandrel 28 and a spray apparatus 30. The filter forming mandrel 28 can be dimensioned as appropriate for any particular filter size and configuration and can be formed of any suitable metallic or polymeric material. In some instances, the filter forming mandrel 28 can have a release coating applied thereto in order to facilitate removal of a finished filter membrane 12.

The spray apparatus 30 can generically represent any suitable spraying apparatus that can be configured to provide appropriately sized particles of whichever polymeric material is being applied. In some instances, the spray apparatus 30 can provide particle sizes in the range of about 5 μm to about 100 μm and more particularly about 15 μm to about 60 μm when spraying suitable materials such as polyurethanes.

In FIG. 4, a first layer 32 has been sprayed onto the filter forming mandrel 28. In some instances, the first layer 32 can be a base layer while in other cases the first layer 32 can be a hydrophilic layer. A second layer 34 can subsequently be applied, as shown schematically in FIG. 5. If the first layer 32 is a base layer, the second layer 34 can be a hydrophilic layer. Conversely, if the first layer is a hydrophilic layer, then the second layer 34 can be a base layer. While not expressly illustrated, additional layers can also be applied. For example, in some cases it can be useful to have a hydrophilic layer on an inside surface of the base layer as well as on the outside surface of the base layer.

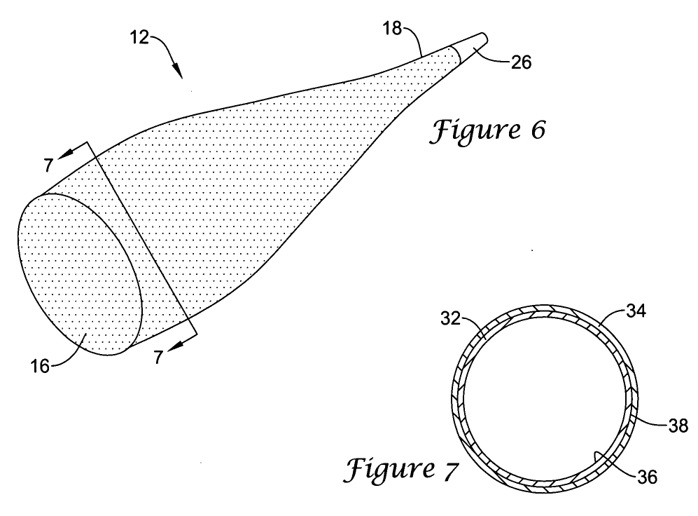

FIG. 6 is a perspective view of the finished filter membrane 12. FIG. 7 is a cross-section illustrating the multi-layer construction of the filter membrane 12. FIG. 7 shows first layer 32 and second layer 34, although additional layers are permissible as discussed above. Merely for illustrative purposes, the first layer 32 can be considered to represent a base layer while the second layer 34 can be considered as representing a hydrophilic layer. The first layer 32 has an inner surface 36 and an outer surface 38. As illustrated, the second layer 34 (representing the hydrophilic layer) is disposed on the outer surface 38 of the first layer 32 (representing the base layer).

In some embodiments, the base layer can be applied to have a thickness that is in the range of about 5 μm to about 50 μm. In particular embodiments, the base layer can have a thickness that is in the range of about 10 μm to about 50 μm. The hydrophilic layer can have a thickness that is in the range of about 0.5 μm to about 8 μm. In particular embodiments, the hydrophilic layer can have a thickness that is in the range of about 0.5 μm to about 5 μm.

In other embodiments, the hydrophilic layer can be disposed on the inner surface 36 of the first layer 32. In some instances, a first hydrophilic layer can be disposed on the inner surface 36 of the first layer 32 while a second hydrophilic layer can be disposed on the outer surface 38 of the first layer 32, assuming of course that the first layer 32 represents a base layer.

The base layer can be formed of any suitable polymeric materials, such as polyether block amide, polybutylene terephthalate/polybutylene oxide copolymers sold under the Hytrel® and Arnitel® trademarks, Nylon 11, Nylon 12, polyurethane, polyethylene terephthalate, polyvinyl chloride, polyethylene naphthalene dicarboxylate, olefin/ionomer copolymers, polybutylene terephthalate, polyethylene naphthalate, ethylene terephthalate, butylene terephthalate, ethylene naphthalate copolymers, polyamide/polyether/polyester, polyamides, aromatic polyamides, polyurethanes, aromatic polyisocyanates, polyamide/polyether, and polyester/polyether block copolymers, among others.

In some embodiments, the base layer can be formed of a polyurethane that absorbs less than about 5 percent of its own weight in water. In some cases, the base layer can be formed from a polycarbonate urethane such as that available commercially under the BIONATE® name.

The hydrophilic layer (or layers) can as discussed above be formed of hydrophilic materials that can absorb from about 2 to about 20 times their own weight in water. The hydrophilic material can be a nonionic material such as the HYDROSLIP® and TECOGEL® materials discussed above. In some embodiments, these materials can be particularly useful, as they are readily dissolvable in water/alcohol mixtures to form low viscosity solutions that are easily sprayable. These materials are compatible with materials used to form the base layer and exhibit good adhesion to the base layer.

The hydrophilic material can be an anionic material such as a sulfonated polyurethane or a carboxylated polyurethane. A polyurethane can be formed from monomers, chain extenders or oligomers that include a desired functional group that can provide a polymer with desired anionomer character. In some embodiments, a diamine disulfonic acid can be used as a chain extender in synthesizing a sulfonated polyurethane. In particular, a sulfonated polyurethane can be produced using 4,4′-diamino-2,2′-biphenyl disulfonic acid as a chain extender. Alternatively, a polyurethane can be formed, and desired functional groups such as sulfonate groups can subsequently be added via a grafting reaction.

An illustrative but non-limiting method of forming a sulfonated polyurethane is described herein. A polyurethane can be formed by first reacting a diisocyanate with an active hydrogen source to create a polyurethane backbone, and subsequently substituting a desired functional group. For example, a desirable functional group includes a sulfonate functional group. A sulfonate functional group can be added to a polyurethane backbone by reacting the polyurethane with a molecule bearing the desired substituent. An example of a desired substituent is a pendent propyl sulfonate group.

One way of adding this functional group is to react the polyurethane backbone with propane sulftone, which is also known as 1,2-oxathiolane-2,2-dione and has the following structure:

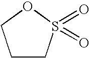

Polyurethanes suitable for use in the present invention can also include copolymers formed by reacting a diisocyanate, a diol and an ether. In particular, a suitable polyurethane can be formed by reacting methylene bis-(p-phenyl isocyanate) (MDI), N-methyldiethanolamine (MDEA) and poly(tetra-methylene oxide) (PTMO). Alternatively, 1,4-butanediol can be used as a chain extender in place of the MDEA.

A carboxylated polyurethane can be formed in a variety of ways. An illustrative but non-limiting method is described herein. A polyurethane bearing pendent carboxyl groups can be formed by reacting an aliphatic diisocyanate, a diol component and a carboxylic acid. In particular, a carboxylated polyurethane polymer can be produced as a reaction product of a diol component, an aliphatic diisocyanate, water and a 2,2-di-(hydroxymethyl) alkanoic acid. Alternatively, an amount of amine, such as diglycolamine can be used for at least a portion of the water in the reaction to form the reaction product.

The diol component can include a polyoxyalkylene diol, such as polyoxyethylene diol having a molecular weight of from about 400 to about 20,000, polyoxypropylene diol having a number average molecular weight of about 200 to about 2,500, block copolymers of ethylene oxide and propylene oxide having a molecular weight of about 1,000 to about 9,000 and polyoxytetramethylene diol having a number average molecular weight of about 200 to about 4,000.

The polyurethane can include a low molecular weight alkylene glycol such as ethylene glycol, propylene glycol, 2-ethyl-1-1,3-hexanediol, tripropylene glycol, triethylene glycol, 2,-4-pentane diol, 2-methyl-1,3-propanediol, 2,-methyl-1,3-pentanediol, cyclohexanediol, cyclohexanedimethanol, dipropylene glycol, diethylene glycol, and mixtures thereof.

An amine can be used in the reaction for at least a portion of the water in the reaction mixture. The amine can be diglycolamine, although other amines such as ethylene diamine, propylene diamine, monoethanolamine, diglycolamine, and propylene diamine can also be used.

The diisocyanate used can include both aliphatic and aromatic types and mixtures thereof. An example of a suitable isocyanate is methylene bis(cyclohexyl-4-isocyanate). Other examples of diisocyanates are trimethyl hexamethylene diisocyanate and isophorone diisocyanate. Representative examples of aliphatic diisocyanates include tetramethylene diisocyanate, hexamethylene diisocyanate, trimethylene diisocyanate, trimethylene hexamethylene diisocyanate, cyclohexyl 1,2-diisocyanate, cyclohexylene 1,4-diisocyanate, and aromatic diisocyanates such as 2,4-toluene diisocyanates and 2,6-toluene diisocyanates.

The invention should not be considered limited to the particular examples described above, but rather should be understood to cover all aspects of the invention as set out in the attached claims. Various modifications, equivalent processes, as well as numerous structures to which the invention can be applicable will be readily apparent to those of skill in the art upon review of the instant specification.

We claim:

1. A method of forming an intravascular filter comprising a polymeric base layer and a hydrophilic layer disposed on the base layer, the method comprising steps of:
providing a filter forming mandrel;
spraying the filter forming mandrel with a hydrophilic polymer to form the hydrophilic layer; and
spraying the hydrophilic layer with a base polymer to form the base layer.

2. The method of claim 1, further comprising a subsequent step of laser drilling the intravascular filter to provide a plurality of apertures therethrough.

3. The method of claim 1, further comprising a step of drying the hydrophilic layer prior to spraying the hydrophilic layer with the base polymer.

4. The method of claim 1, further comprising a step of spraying the polymeric base layer with a hydrophilic polymer to form a second hydrophilic layer.

5. The method of claim 1, wherein spraying a hydrophilic polymer comprises spraying a hydrophilic polyurethane.

6. The method of claim 1, wherein spraying a hydrophilic polymer comprises spraying a nonionic hydrophilic polyurethane.

7. The method of claim 1, wherein spraying a hydrophilic polymer comprises spraying a nonionic polyether polyurethane.

8. The method of claim 1, wherein spraying a hydrophilic polymer comprises spraying a nonionic aliphatic polyether polyurethane.

9. The method of claim 1, wherein spraying a hydrophilic polymer comprises spraying an anionic polyurethane.

10. The method of claim 1, wherein spraying a hydrophilic polymer comprises spraying a sulfonated polyurethane or a carboxylated polyurethane.

11. The method of claim 1, wherein spraying a base polymer comprises spraying a polyurethane.

12. The method of claim 1, wherein spraying a base polymer comprises spraying a polycarbonate urethane.

13. A method of forming an intravascular filter comprising a polymeric base layer and a hydrophilic layer disposed on the base layer, the method comprising steps of:
providing a filter forming mandrel;
spraying the filter forming mandrel with a base polymer to form the base layer; and
spraying the base layer with a hydrophilic polymer to form the hydrophilic layer.

14. The method of claim 13, further comprising a subsequent step of laser drilling the intravascular filter to provide a plurality of apertures therethrough.

15. The method of claim 13, further comprising a step of drying the base layer prior to spraying the base layer with the hydrophilic polymer.

16. The method of claim 13, wherein spraying a base polymer comprises spraying a polyurethane.

17. The method of claim 13, wherein spraying a base polymer comprises spraying a polycarbonate urethane.

18. The method of claim 13, wherein spraying a hydrophilic polymer comprises spraying a hydrophilic polyurethane.

19. The method of claim 13, wherein spraying a hydrophilic polymer comprises spraying a nonionic hydrophilic polyurethane.

20. The method of claim 13, wherein spraying a hydrophilic polymer comprises spraying a nonionic polyether polyurethane.

21. The method of claim 13, wherein spraying a hydrophilic polymer comprises spraying a nonionic aliphatic polyether polyurethane.

22. The method of claim 13, wherein spraying a hydrophilic polymer comprises spraying an anionic polyurethane.

23. The method of claim 13, wherein spraying a hydrophilic polymer comprises spraying a sulfonated polyurethane or a carboxylated polyurethane.

24. An intravascular filter, comprising:
a filter membrane having an inner surface and an outer surface; and
a hydrophilic polyurethane coating disposed on the filter membrane.

25. The intravascular filter of claim 24, wherein the hydrophilic polyurethane coating is disposed on the inner surface of the filter membrane.

26. The intravascular filter of claim 24, wherein the hydrophilic polyurethane coating is disposed on the outer surface of the filter membrane.

27. The intravascular filter of claim 24, wherein the hydrophilic polyurethane coating is capable of absorbing from about 2 to about 20 times its own weight in water.

28. The intravascular filter of claim 24, wherein the hydrophilic polyurethane coating comprises a nonionic hydrophilic polyurethane.

29. The intravascular filter of claim 24, wherein the hydrophilic polyurethane coating comprises a nonionic polyether polyurethane.

30. The intravascular filter of claim 24, wherein the hydrophilic polyurethane coating comprises a nonionic aliphatic polyether polyurethane.

31. The intravascular filter of claim 24, wherein the hydrophilic polyurethane coating comprises an anionic polyurethane.

32. The intravascular filter of claim 24, wherein the hydrophilic polyurethane coating comprises a sulfonated polyurethane or a carboxylated polyurethane.

33. The intravascular filter of claim 24, wherein the filter membrane comprises polyurethane adapted to absorb no more than about 5 percent of its weight in water.

34. The intravascular filter of claim 24, wherein the filter membrane comprises a polycarbonate urethane.

35. The intravascular filter of claim 24, wherein the filter membrane has an average thickness that is in the range of about 5 μm to about 50 μm.

36. The intravascular filter of claim 24, wherein the hydrophilic polyurethane coating has an average thickness that is in the range of about 0.5 μm to about 8 μm.
